(19) United States
(12) Patent Application Publication
(10) Pub. No.: US 2003/0073139 A1
(43) Pub. Date: Apr. 17, 2003

(54) Devices and methods for verifying measurement of analytes by raman spectroscopy and surface plasmon resonance

(76) Inventors: David I. Kreimer, Berkeley, CA (US); Oleg A. Yevin, Oakland, CA (US)

Correspondence Address: FLIESLER DUBB MEYER & LOVEJOY, LLP, FOUR EMBARCADERO CENTER, SUITE 400, SAN FRANCISCO, CA, 94111, US

(21) Appl. No.: 10/245,922

(22) Filed: Sep. 18, 2002

Related U.S. Application Data
(60) Provisional application No. 60/323,981, filed on Sep. 21, 2001.

(51) Int. Cl. G01N 33/53; G01N 33/542; C12M 1/34; G01J 3/44

(52) U.S. Cl. 435/7.9; 435/287.2; 356/301

(57) ABSTRACT

Embodiments of devices and methods are provided that permit validation of analyte detection using both surface enhanced Raman spectroscopy (SERS) and surface plasmon resonance (SPR). In specific embodiments, a substrate having a surface suitable for SPR is provided, along with a source of electromagnetic radiation to interact with the surface and thereby elicit surface plasmon resonance characteristic of the analyte under study. In some embodiments, surface enhancing structures are also provided on the substrate, and analytes under study are associated with enhancing structures. Another source of electromagnetic radiation is directed at the analyte on the enhancing structures to produce surface enhanced Raman scattering. In certain embodiments, data obtained by these two methods are compared, thereby providing an internally consistent and self-validating method for analyte detection.

### RELATED APPLICATION

This application claims priority under 35 U.S.C. §119(e) to U.S. Provisional Patent Application Serial No. 60/323,981, filed Sep. 21, 2001. This application also claims priority to U.S. Provisional Patent Application Serial No. 60/156,195, filed Sep. 27, 1999, now abandoned, and to United States Utility patent application Ser. Nos. 09/670,453, filed Sep. 26, 2000, 09/815,909, filed Mar. 23, 2001, and 09/925,189 filed Aug. 8, 2001. Each of the above patent applications is incorporated herein fully by reference.

### BACKGROUND

Detection and quantification of analytes in complex mixtures of substances is a substantial component of medical, environmental and industrial processes. However, detection and quantification remains laborious, time consuming and expensive. Generally, measurements and their methods are designed specifically for the analyte to be measured, processes that can be challenging and expensive. Modem drug discovery is based in part, on high throughput screening (HTS) of candidate molecules. For many prior art methods, labeling of the analyte is required, and for detection of nucleic acids, polymerase chain reaction (PCR) is often used. Unfortunately, labeling and PCR methods are time consuming, expensive and can lead to errors.

Recently, new methods have been developed that can eliminate one or more of the time consuming steps previously considered to be necessary. For example, Raman spectroscopy can be used to detect analytes directly, without the need for labeling or PCR steps. Examples of Raman methods are described in United States Patent Application titled “Particle Structures with Receptors for Analyte Detection”, Ser. No. 09/670,453 and United States Patent Application titled “Addressable Arrays Using Morphology Dependent Resonance for Analyte Detection”, Ser. No. 09/669,369. Both of these patent applications are herein incorporated fully by reference.

Spectroscopic methods for analyte detection can exhibit “false positives”, in which a signal is interpreted to be from an analyte of interest, but is actually derived from another species. Decreasing false positives can be accomplished using receptor-mediated analyte binding methods, for example, those in U.S. Patent Ser. Nos. 09/670,453 and 09/669,369.

### SUMMARY

An object of this invention is to decrease the frequency of false positive results in spectrographic analysis. In general, methods and devices of this invention can reduce the incidence of false positives by providing verification of results obtained using one method by making measurements of the same sample using a different method.

This invention includes methods and devices for verifying results obtained using resonance spectroscopic methods. In certain embodiments, a sample is analyzed simultaneously using Surface Enhanced Raman Spectroscopy (SERS) and surface plasmon resonance (SPR). By subjecting the same sample to two different analytical methods, the results obtained using each method can be compared to those results obtained using the other method, thereby providing internal validation of the results obtained. Because SERS and SPR methods have technical features in common, the use of those two methods can be accomplished easily and relatively inexpensively. Moreover, because both SERS and SPR permit direct detection of analytes by optical means, pre-treatment of samples may not be required. Because both SERS and SPR are optical methods, they can be accomplished rapidly and information can be stored for further comparison.

### BRIEF DESCRIPTION OF THE DRAWINGS

FIG. 1 is a drawing depicting a process for analyzing analytes using Raman spectroscopy and surface plasmon resonance.

FIG. 2a depicts an embodiment of this invention for detecting an analyte using Raman spectroscopy and surface plasmon resonance on different areas of a biochip.

FIG. 2b depicts an embodiment of this invention for detecting an analyte using Raman spectroscopy and surface plasmon resonance on the same area of a biochip.

FIG. 3 depicts an embodiment of this invention for simultaneous SERS and SPR analysis of an analyte on a biochip.

### DETAILED DESCRIPTION

This invention includes methods for verifying SERS and SPR results obtained for the same sample. In some cases, the measurements can be made simultaneously A biochip can be prepared having a metal layer suitable for SPR measurements. Methods for preparing such surfaces are known in the art. In certain aspects of this invention, the metal layer can be applied to a surface of a prism. A light source generates a beam that can enter the prism, interact with the metal layer, and thereby generate surface plasmon resonance in the metal layer. Analytes present near this metal layer can be detected and quantified by the production of spectral features characteristic of the analyte present. To increase the relative amount of a desired analyte present, a receptor can be applied to the metal layer. Analytes that can readily associate with the receptor become relatively concentrated near the metal layer, thereby increasing the intensity of SPR signals. SPR signals can be captured by a light detector, and the relative intensity and angle of the output beam can be converted into signals (e.g., electrical or optical) which can be transmitted to a computer or a storage device for analysis.

Surface enhanced Raman spectroscopy (SERS) can be carried using methods and enhancing structures can be prepared using methods described in U.S. Utility patent application Ser. Nos. 09/670,453, filed Sep. 26, 2000, 09/815,909, filed Mar. 23, 2001, and 09/925,189 filed Aug. 8, 2001, incorporated herein fully by reference. Alternatively, surface enhancing conditions can be provided using roughened metal surfaces as described in U.S. Pat. No. 5,122,127, incorporated herein fully by reference. In some embodiments, SERS can be carried out on the same sample as used for SPR measurements, either simultaneously (by use of a beam splitter) to divide the source light beam into two beams, one for SPR, and another for SERS measurements. Alternatively, two independent light sources can be used, and in other embodiments, a source beam can first be used to perform SERS measurements, and subsequently, to perform SPR measurements. Of course, one can reverse the order of measurements if desired.

In some embodiments, analyte receptors can be provided to increase the selectivity of the assay system. Analyte receptors for Raman spectroscopy are described in U.S. patent application Ser. Nos. 09/670,453, filed Sep. 26, 2000, 09/815,909, filed Mar. 23, 2001, and 09/925,189 filed Aug. 8, 2001, incorporated herein fully by reference. Receptors can be attached to the SPR surface, to enhancing structures, or to both SPR surfaces and SERS enhancing structures. Additionally, the selectivity and sensitivity of analyte detection can be improved by the use of a passivating agent, such as mercaptoethanol, mercaptohexanol or other mercaptoalkanol. Passivation methods are described in U.S. patent application Ser. No. 09/925,189, herein incorporated fully by reference.

Detection can be carried out for a variety of analytes, including by way of example only, proteins, nucleic acids, lipids, carbohydrates, low molecular weight compounds of biomedical significance present in organisms such as mammals, fungi, bacteria and viruses, and cellular organelles from eukaryotic organisms. Moreover, complexes of biomolecules can be analyzed using the verified methods of this invention. Detection can be carried out using either a single detector, or using a number of detectors simultaneously. In certain embodiments, a filter-based spectrographic analysis system can be used. Such systems are described in U.S. patent application Ser. No. 09/939,887, incorporated herein fully by reference.

The results of SERS and SPR measurements can be stored in a database, computer, or displayed on a computer monitor or a print out. The information obtained can be compared using programs to decrease the incidence of false positive results.

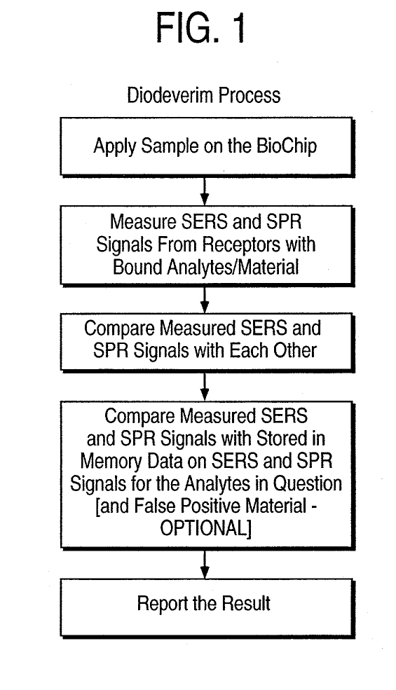

FIG. 1 depicts a schematic drawing of a process of some embodiments for direct optical detection, verification and measurement (herein termed a “Diodeverim Process” or “DP”). A sample to be analyzed is applied to a biochip, SERS and SPR signals generated by analytes in the sample are collected and stored. The stored signals are compared with each other, and possibly with data previously stored in memory for either the analytes of interest, or for other, contaminating materials which maybe responsible for false positive results. The previous step is optional. Once comparisons of the results obtained by SERS and SPR are made, a report of the results can be displayed, stored, or further used to process the information.

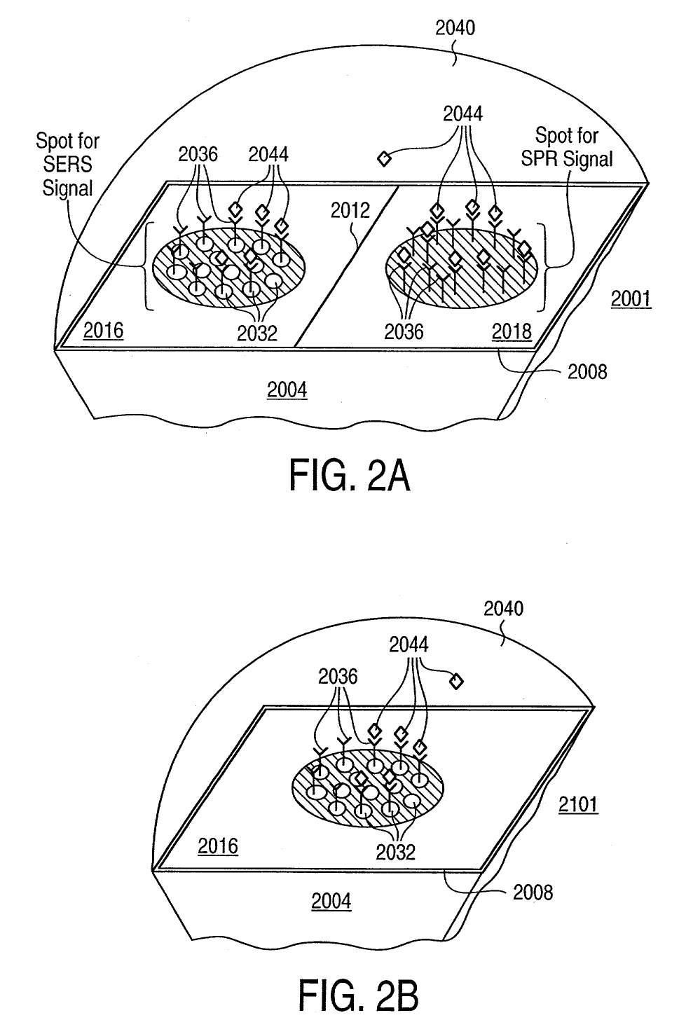

FIGS. 2a and 2b depict embodiments of this invention. FIG. 2a depicts an embodiment 2001 having two different areas, one for SERS measurements, an another for SPR measurements. Biochip 2001 comprising a prism 2004 (only the top part of the prism is shown), and having a metal layer 2008 thereon. Prism 2004 can be produced using methods known in the art or purchased commercially (e.g., from Biocore Inc.). Metal layer 2008 is selected to provide surface plasmon resonance conditions. The surface of the prism is shown being divided into two areas by a separator line 2012, which, in this case, is an area devoid of metal. Area 2016 is depicted as having no metal layer 2008 thereon. However, area 2016 has particle structures 2032 (e.g., nanoparticles, fractal structures or other enhancing structures) that can provide enhancing conditions for SERS measurements. Receptors 2036 are associated with enhancing structures 2032, and analytes 2044 are shown associated with or binding to receptors 2036. Area 2018 is an area having a metal layer 2008 thereon, for SPR measurements. Receptors 2036 are depicted associated with metal layer 2008 of area 2018, and analytes 2044 are shown associated with receptors 2036 and free in solution in drop 2040, which is sufficiently large to expose analytes to both areas 2016 and 2018.

To use a device as shown in FIG. 2a, area 2016 is illuminated with an incident beam of electromagnetic radiation sufficient to produce a SERS signal from analytes present near the enhancing structures 2032. Simultaneously or subsequently, area 2018 is illuminated with an incident beam of electromagnetic radiation sufficient to produce a SPR signal from analytes present near the metal layer 2008.

FIG. 2b depicts a device for measuring SERS and SPR signals from the same spot, area 2016 of a biochip. Biochip 2001 comprises prism 2004 (only part of the prism is shown) having a layer of metal 2008 thereon. A portion 2016 of the biochip has enhancing structures 2032 thereon, and receptors 2036 are associated with enhancing structures 2032. Analyte molecules 2044 are shown associated with receptors 2036 and are also free in solution in drop 2040. When exposed to SERS and/or SPR conditions, the analytes produce a spectral feature characteristic of the analyte under study. One advantage of the instrumentation, methods and devices of this invention is that the SPR and SERS detectors can be simple in design. Many detector elements are common to both SERS and SPR instruments.

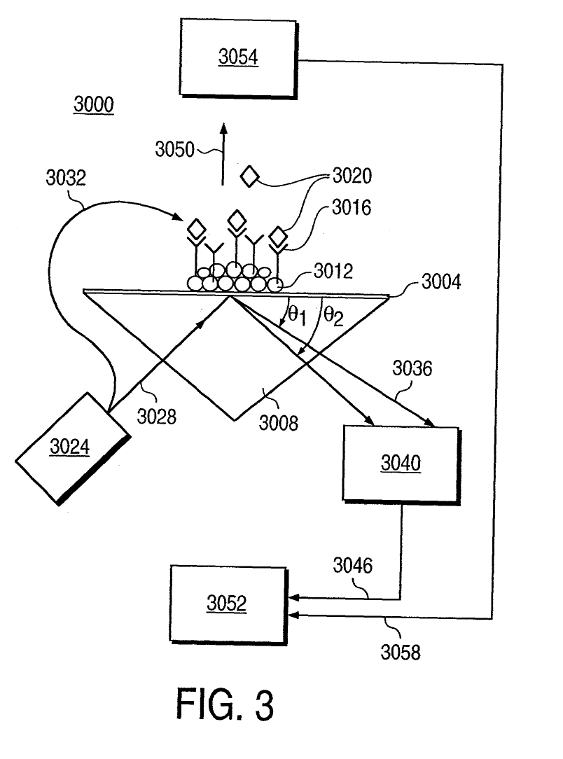

FIG. 3 shows a device 3000 for detecting and verifying measurements of analytes using SERS and SPR methods. A layer of metal 3004 is on a prism 3008. Enhancing structures 3012 are optionally present on surface 3004 and have receptors 3016 attached thereto. If enhancing structures 3012 are present, enhanced Raman signals can be produced. If no enhancing are present, receptors 3016 can be attached directly to the surface of metal layer 3004. Analyte molecules 3020 are show associated with receptors 3016 and free in solution. Light source 3024 produces beams 3028 and 3032. Beam 3028 is directed through prism 3008 an impinges on the underside of surface 3004, generating surface plasmon resonance. Beams of light 3036 leaving surface 3004 have angles θ1 and θ2, which are dependent upon the presence of analytes 3020 associated with surface 3004. Beams 3036 are detected by SPR detector 3040 and the information obtained is transmitted via signal carrier 3046 to computer 3052. Beam 3032 is directed toward the upper surface of surface 3004. Raman signals 3050 produced by analytes 3020 associated with receptors 3016, particles 3012 on surface 3004 are detected by Raman detector 3054. Signals from Raman detector 3054 are transmitted via signal carrier 3058 to computer 3052. The signals produced by SPR detector 3040 and Raman detector 3054 are compared and can be displayed on a screen of computer 3052 or on a printer (not shown) or directed to a data bank (not shown) having trusted computing spaces (not shown).

### INDUSTRIAL APPLICABILITY

This invention includes methods for detecting analytes in biological, environmental and industrial samples, and for verifying results obtained by providing two optical detection methods and comparing the results obtained from the optical detection methods.

We claim:

1. A method for detecting an analyte, comprising the steps of:
(a) providing a surface adapted for SERS and SPR measurements;
(b) providing at least one analyte associated with said surface; and
(c) detecting at least one SPR spectral feature and at least one SERS spectral feature characteristic of said analyte.

2. The method of claim 1, wherein said surface has at least one enhancing structure thereon.

3. The method of claim 1, wherein said surface has a metal layer thereon.

4. The method of claim 2, wherein said enhancing structure comprises a nanoparticle structure.

5. The method of claim 4, wherein said nanoparticle structure is a fractal structure.

6. The method of claim 2, wherein said enhancing structure comprises a metal.

7. The method of claim 6, wherein said metal is selected from the group consisting of gold and silver.

8. The method of claim 3, wherein said metal layer comprises a metal selected from the group consisting of gold and silver.

9. A device for detecting an analyte, comprising:
a first surface adapted to enhance Raman signals;
a second surface adapted to provide surface plasmon resonance;
a source of electromagnetic radiation;
a SERS signal detector associated with said first surface; and
a SPR signal detector associated with said second surface.

10. The device of claim 9, further comprising means for comparing said SERS signal and said SPR signal.

11. A device for detecting an analyte, comprising:
a prism having a surface having at least two areas thereon;
one of said areas having a metal layer thereon adapted to provide surface plasmon resonance conditions;
another of said areas having enhancing structures adapted to provide surface enhanced Raman spectroscopic conditions;
at least one analyte receptor associated with each of said areas; and
at least one detector associated with each of said areas.

12. The device of claim 11, further comprising means for storing signals produced by each of said detectors.

13. A system for detecting an analyte, comprising:
a first surface adapted to enhance Raman signals;
a second surface adapted to provide surface plasmon resonance;
a source of electromagnetic radiation associated with each of said first and second surfaces;
a SERS signal detector associated with said first surface;
a SPR signal detector associated with said second surface; and
means for comparing said SERS signal and said SPR signal.

14. A system for detecting an analyte, comprising:
a surface having means for enhancing Raman signals and means for providing surface plasmon resonance;
a source of electromagnetic radiation;
means for detecting a SERS signal;
means for detecting a SPR signal; and
means for comparing said SERS signal and said SPR signal.

15. The method of claim 1, further comprising binding an analyte receptor to at least one of said surface and said enhancing structure.

16. The device of claim 9, further comprising an analyte receptor associated with at least one of said first surface and said second surface.

17. The system of claim 13, further comprising an analyte receptor associated with at least one of said first surface and said second surface.
